(19) United States
(12) Patent Application Publication
(10) Pub. No.: US 2022/0406425 A1
(43) Pub. Date: Dec. 22, 2022

(54) Incremental Query Effect Management

(71) Applicant: 3M INNOVATIVE PROPERTIES COMPANY, St. Paul, MN (US)

(72) Inventors: Jeffrey S. Seese, Hanover, PA (US); Julie A. Salomon, Atlanta, GA (US); Mindy W. Kozlowski, New Market, MD (US); Julie L. Imburgia, Draper (UA); Christine F. Bayless, St. George, UT (US); Glen R. Keller, Hanover, MD (US); Benjamin J. Raubenolt, Northfield, OH (US); Michael J. Polly, Brecksville, OH (US); Edward A. Mai, Haymarket, VA (US)

(21) Appl. No.: 17/755,111

(22) PCT Filed: Nov. 20, 2020
(86) PCT No.: PCT/IB2020/060976

§ 371 Date: Apr. 21, 2022

Related U.S. Application Data
(63) Continuation of application No. 62/938,534, filed on Nov. 21, 2019.

(51) Int. Cl. G16H 15/00; G16H 50/20; G16H 10/60

(52) U.S. Cl. CPC G16H 15/00; G16H 50/20; G16H 10/60

(57) ABSTRACT

A computer implemented method includes generating a first interface view showing a first diagnosis for a patient during a patient encounter with a healthcare provider, receiving a first query in response to the first interface view, the query soliciting further specificity with respect to the first diagnosis, making the query available to the healthcare provider, receiving a first response providing further specificity with respect to the first diagnosis, receiving an updated code in response to the first response, storing the code, receiving one or more further updated codes specified by the CDI specialist in response to one or more queries and responses based on further diagnoses, storing the one or more further codes, and generating an incremental query impact summary based on the updated code and one or more further updated codes to illustrate the effect of each stored updated codes in response to queries.

### BACKGROUND

Documenting patient encounters is typically done using tools within an Electronic Health Record (EHR) application. A clinical documentation integrity specialist (CDI) is responsible for ensuring that EHR documentation is clinically accurate and complete. CDI specialists typically use an application downstream from the EHR which provides clinical review tools. A medical encounter refers to an interaction between a patient and healthcare provider, such as a patient visit to a hospital. This can range from a simple diagnoses report from a clinician, to a paper trail that may include admission diagnoses, radiology reports, progress and nursing notes, and discharge summary span over the duration of days or weeks.

Some medical related applications include functions to help translate unstructured information about diagnoses, treatments, procedures, medications and equipment into alphanumeric codes, such as International Classification of Diseases (ICD) codes, or Current Procedural Terminology (CPT) codes, for billing or insurance purposes. To correctly interpret this information, experienced professional CDI specialists are often involved in the process of medical coding. CDI specialists are typically educated and experienced clinicians, such as RNs and BSNs. They are responsible for ensuring the highest and most accurate specificity of a diagnosis and ensuring that all indicated diagnoses are documented. This can result in a more accurate code applied by a medical coder downstream. Mislabeled coding can lead to increased expense in treatment as well as lower reimbursement. Mislabeled coding can also lead to an inaccurate representation of a patient population and severity of illness.

The medical related application may show a diagnosis, however, a CDI specialist may be looking for a more specific diagnosis or a potentially missing diagnosis. The CDI specialist user may generate a query for the healthcare provider such as the clinician or doctor to specify the condition in more detail. The received answer or response may lead to the creation of more complete and accurate provider documentation, which can lead to a more accurate code.

Many times, a change in the patient's health during a medical encounter may lead to a change in diagnoses, and hence a change in coding. The effect of the change may be reflected in a set of final diagnosis codes that may not reflect the journey of the patient during the encounter and may also not reflect the efficiency of the specialist in reviewing the clinical content and querying to enhance the integrity of the documentation during the encounter to support continuity of care.

### SUMMARY

A computer implemented method includes generating a first interface view showing a first diagnosis for a patient during a patient encounter with a healthcare provider, receiving a first query from a CDI specialist in response to the first interface view, the query soliciting further specificity with respect to the first diagnosis, making the query available to the healthcare provider, receiving a first response providing further specificity with respect to the first diagnosis, receiving an updated code specified by the CDI specialist in response to the first response, storing the code, receiving one or more further updated codes specified by the CDI specialist in response to one or more queries and responses based on further diagnoses, storing the one or more further codes, and generating an incremental query impact summary based on the updated code and one or more further updated codes to illustrate the effect of each stored updated codes in response to queries.

The method may be used to document a series of incremental transactions in further embodiments to track and measure the performance of such incremental transactions.

### BRIEF DESCRIPTION OF THE DRAWINGS

FIG. 1 is a block diagram view of a system for keeping track of transactions and showing incremental effects of intermediate transactions according to an example embodiment.

FIG. 2 is a flowchart illustrating a computer implemented method of generating a summary showing the effects of the incremental queries according to an example embodiment.

FIG. 3 is a create query interface generated by application in response to selection of query according to an example embodiment.

FIG. 4 is a pop-up view displayed in response to selection of a Baseline Dx field according to an example embodiment.

FIG. 5 is a pop-up view displayed in response to selection of an Actual Result field according to an example embodiment according to an example embodiment.

FIG. 6 is a query grid interface screen that lists queries and their status according to an example embodiment.

FIG. 7 is an interface view corresponding to an impact review showing the effects of incremental queries during progression of a case according to an example embodiment.

FIG. 8 is an interface view showing an example working summary of the effects of the various incremental impact summaries that the system 100 is configured to perform according to an example embodiment.

FIGS. 9A and 9B are a user interface corresponding to the impact review with different portions of the view 900 expanded according to an example embodiment.

FIG. 10 is a block schematic diagram of a computer system to implement one or more example embodiments.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized, and that structural, logical, and electrical changes may be made without departing from the scope of the present invention. The following description of example embodiments is, therefore, not to be taken in a limited sense, and the scope of the present invention is defined by the appended claims.

The functions or algorithms described herein may be implemented in software in one embodiment. The software may consist of computer executable instructions stored on computer readable media or computer readable storage device such as one or more non-transitory memories or other type of hardware-based storage devices, either local or networked. Further, such functions correspond to modules, which may be software, hardware, firmware or any combination thereof. Multiple functions may be performed in one or more modules as desired, and the embodiments described are merely examples. The software may be executed on a digital signal processor, ASIC, microprocessor, or other type of processor operating on a computer system, such as a personal computer, server or other computer system, turning such computer system into a specifically programmed machine.

The functionality can be configured to perform an operation using, for instance, software, hardware, firmware, or the like. For example, the phrase “configured to” can refer to a logic circuit structure of a hardware element that is to implement the associated functionality. The phrase “configured to” can also refer to a logic circuit structure of a hardware element that is to implement the coding design of associated functionality of firmware or software. The term “module” refers to a structural element that can be implemented using any suitable hardware (e.g., a processor, among others), software (e.g., an application, among others), firmware, or any combination of hardware, software, and firmware. The term, “logic” encompasses any functionality for performing a task. For instance, each operation illustrated in the flowcharts corresponds to logic for performing that operation. An operation can be performed using, software, hardware, firmware, or the like. The terms, “component,” “system,” and the like may refer to computer-related entities, hardware, and software in execution, firmware, or combination thereof. A component may be a process running on a processor, an object, an executable, a program, a function, a subroutine, a computer, or a combination of software and hardware. The term, “processor,” may refer to a hardware component, such as a processing unit of a computer system.

Furthermore, the claimed subject matter may be implemented as a method, apparatus, or article of manufacture using standard programming and engineering techniques to produce software, firmware, hardware, or any combination thereof to control a computing device to implement the disclosed subject matter. The term, “article of manufacture,” as used herein is intended to encompass a computer program accessible from any computer-readable storage device or media. Computer-readable storage media can include, but are not limited to, magnetic storage devices, e.g., hard disk, floppy disk, magnetic strips, optical disk, compact disk (CD), digital versatile disk (DVD), smart cards, flash memory devices, among others. In contrast, computer-readable media, i.e., not storage media, may additionally include communication media such as transmission media for wireless signals and the like.

An improved medical coding application facilitates coding of patient encounters that facilitates querying health care providers to obtain more accurate, updated codes for the patient encounter. The application further stores data corresponding to the queries to track the effect each query has on improved coding. The application further generates an incremental query impact summary to show the effect of each query on a weight for changes in coding in response to each query. The application can also be configured to generate an incremental query impact summary to show anticipated impacts should a response be provided and missed opportunity impacts should a query go unanswered. The summary allows documentation departments to better measure the quality and completeness of documents. Updated severity of illness and risk of mortality for patients may also be calculated and shown in the summary, along with changes in reimbursement for services provided during the patient encounter.

The following acronyms and terms that may be used in drawings and following text are provided to facilitate understanding:

CDI—Clinical Documentation Integrity. Also used to refer to an application implementing incremental query summaries and facilitating clinical documentation review and coding.

DRG—Diagnosis Related Group

APR DRG—All Patients Refined Diagnosis Related Groups

DRG Weight—Each DRG weight represents the average resources required to care for cases in that particular DRG, relative to the average resources used to treat cases in all DRGs.

CAPD—Computer Assisted Physician Documentation

CC—Complication or Comorbidity

CDI Impact

Dx—diagnosis

GMLOS—Geometric Length of Stay

HAC—Hospital Acquired Condition

HCC—Hierarchical Condition Category

PDx—Principal Diagnosis

MCC—Major Complication or Comorbidity

MS-DRG—Medicare Severity-Diagnosis Related Group

PSI—Patient Safety Indicator

PPC—Potentially Preventable Complication

Px—Procedure

ROM—APR DRG Risk of Mortality Subclass

SOI—APR DRG Severity of Illness subclass.

WT DRG weight.

Estimate of Reimb—Reimbursement value is returned by a CRS (Coding and Reimbursement System) which takes into account factors like total charges, transfer payments, and pass-through payments. While total reimbursement accurately reflects payment, it can result in calculation of financial impact differences when you don't expect to see them, even if the DRG does not change.

Analysis Reimb—Reimbursement value is returned by a CRS (Coding and Reimbursement System) which excludes visit-specific factors that can affect the reimbursement so that changes in reimbursement result solely from changes in the DRG. A temporary “SummaryQuerylmpact” table is created ‘on the fly’ in the Stored Procedure to produce the Impact Review Summary information with these fields:

Field Name

[Baseline SOI]

[Baseline ROM]

[BaselineWeight]

[FinalSOI]

[FinalROM]

[FinalWeight]

[FinalMCCCount]

[FinalCCCount]

[QueryImpactMCCCC]

[FinancialImpactAmount]

[IsPrincipalDiagnosisImpact]

[MSGrouperVersion]

[MSDRG]

[MSDRGDescription]

[MSWeight]

[MSReimbursement]

[MSAnalysisReimbursement]

[APRGrouperVersion]

[APRDRG]

[APRDRGDescription]

[APRSOI]

[APRROM]

[APRWeight]

[APRReimbursement]

[APRAnalysisReimbursement]

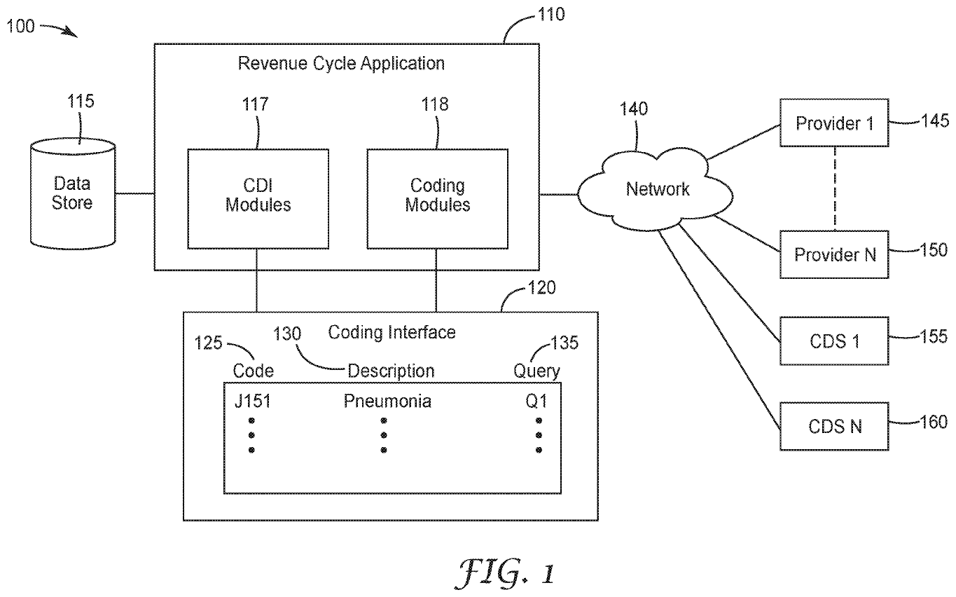

FIG. 1 is a block diagram view of a system 100 that includes an application 110 for use in keeping track of transactions and showing incremental effects of intermediate transactions. The system 100 can not only calculate the effects of intermediate transactions upon completion of a final transaction, but calculate the anticipated effects of the final transaction being completed and calculate the opportunity costs where a final transaction never materializes. This allows the system 100 to improve resource utilization generally and can identify areas where, e.g., the provision of medical care can be improved. The system 100 may be used to track negotiations between two parties and the progress made in interactions between the parties to monitor the performance of the negotiators. As the final agreement may change some of the results of intermediate exchanges, tracking the incremental changes is a way to better evaluate the performance of the negotiators. Other applications may also benefit from use of system 100, including medical documentation that includes the coding of patient encounters.

In one embodiment, the application 110 comprises an application for use by a CDI specialist in generating codes corresponding to patient encounters. In one embodiment, application 110 may be part of a system, such as a 3M 360 Encompass System that integrates computer-assisted coding (CAC), clinical documentation improvement (CDI), concurrent quality metrics and analytics into one application to capture, analyze and advance patient information across the care continuum.

Application 110 may access a data store 115. Data store 115 may store data collected by other systems regarding the patient encounter. The data store 115 may include information generated by healthcare providers regarding the patient encounter, including clinical indicators describing the patient and their condition, as well as insurance information, patient identification and demographic information. Application 110 may access such information to generate prompts and other user interfaces for various users, such as clinical documentation specialists indicated 155 and 160. The application 110, via CDI modules 117 and coding modules 118, generates a user interface 120. In the case of a coding application, user interface 120 may include a plurality of codes 125, corresponding descriptions 130, and a corresponding query 135. Note that in some instances, a diagnosis or procedure may be missing, and the query may be generated by the user to obtain more information to enable a diagnosis or procedure to be determined, or if already existing, more specific information.

The application 110 may be part of a larger medical record system or a stand-alone application in various embodiments. The application 110 may be coupled via a network 140 to one or more providers indicated at 145 and 150. When selecting one of the listed queries 135, the user may generate a query to be sent to one of the providers 145, 150 to obtain the further information. The further information may enable the user to generate a more specific code. Each code, when grouped with other codes on the encounter, will generate further information, such as a weight, description, SOI, ROM, PDx, MCC, CC, DRG Grouping, CDI impact, and POA to name a few. This further information may be the result of a combination and interaction of multiple codes. The information better describes the condition of the patient and may also affect the care of the patient as well as the reimbursement that the healthcare provider may receive for treating the patient. Accurate coding can be beneficial to the patient and the healthcare provider.

In prior coding systems, the final codes existing at the time of discharge or the end of the patient encounter is usually used for obtaining payment for the encounter from an insurance company. The accuracy of the coding is important. The performance of the CDI specialist can be difficult to measure, as a diagnosis may change along the way, and the intermediate codes and changes to codes based on incremental queries occurring during the encounter may have been well done, but the final coding can obscure such efforts of coders. Application 110 captures and stores the effects of these incremental queries and provides a summary of the value provided by the incremental queries.

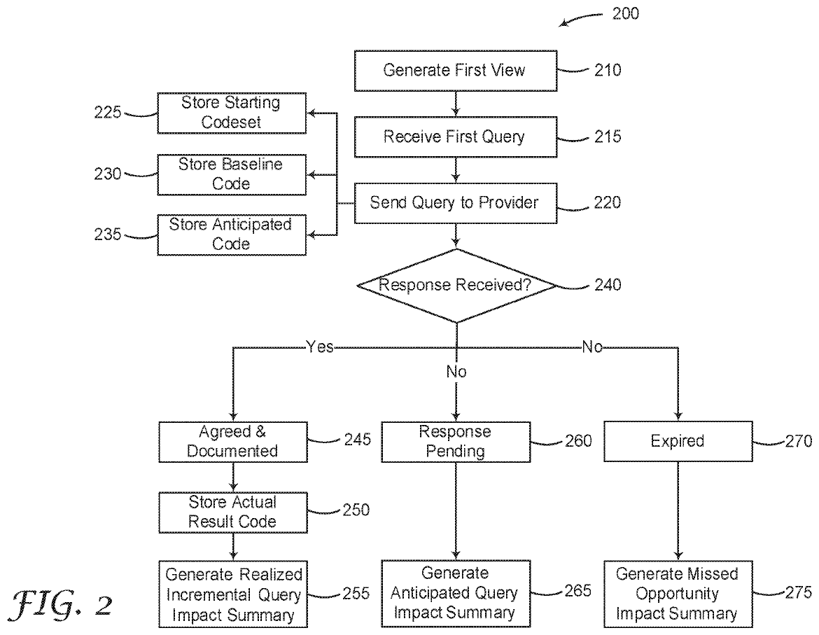

FIG. 2 is a flowchart illustrating a computer implemented method 200 of generating a summary showing the effects of the incremental queries by a coder, also referred to as a user. Method 200 begins with application 110 generating a first interface view at operation 210 showing a first diagnosis for a patient during a patient encounter with a healthcare provider. Information from data store 115 is used to populate the interface. In some implementations, the first diagnosis includes a description and a code. The code may be generated by the application 110 based on medical records in the data store 115 or may be entered by a user based on information displayed, such as the diagnosis provided by the healthcare provider. In some instances, there may be a more specific code that can be associated with the diagnosis. For example, in the case of a diagnosis of respiratory failure, the type and acuity may provide a more specific indication of the patient's condition, such as Acute respiratory failure with hypoxia or with hypercapnia, or chronic respiratory failure with hypoxia. Each of these has more specific associated codes.

At operation 215, a first query may be received via the interface 120 from a CDI specialist in response to the first interface view. The query may be a natural language query soliciting further specificity with respect to the first diagnosis. The query may be manually generated by the CDI specialist as a result of reviewing documentation. The query may even suggest several contextual multiple-choice diagnosis options. At operation 220, the query is made available to the healthcare provider. The query may be made available as an electronic communication, a text message, a fax, or even within an application used by the healthcare provider associated with the patient encounter. The healthcare provider may be a doctor, physician assistant, nurse, or other provider that provided the service to the patient. At operation 225, the starting codeset is stored. This typically occurs when the query is made available to the healthcare provider. For instance, when the query is made available, a unique identifier for the most recent revision of the codeset is stored in datastore 115 along with the query data. This establishes a link between the query and the codeset (described elsewhere in the specification as a “linked codeset”) that the system 100 can use for calculating the impact the query had at the time it was made available to the healthcare provider. It should be appreciated that each time the set of codes associated to a patient encounter changes (added, edited, deleted) and are saved, the new codeset data is stored in datastore 115. Codeset data consists of all diagnosis, procedure codes, and grouper results. Additionally, save event data is additionally stored, including the event date and time, identifier unique to the event, whether CDI or Coding staff performed the save event. MS DRG grouper and APR DRG grouper results data may also include:
Diagnoses—All codes with designation which code is principal and designation if each code was present on admission (POA).
Procedures—All codes.
MS DRG Grouper—MS DRG, MS DRG Weight, MS DRG Estimated Financial Reimbursement.
APR DRG Grouper—APR DRG, APR DRG Weight, Severity of Illness, and Risk of Mortality.

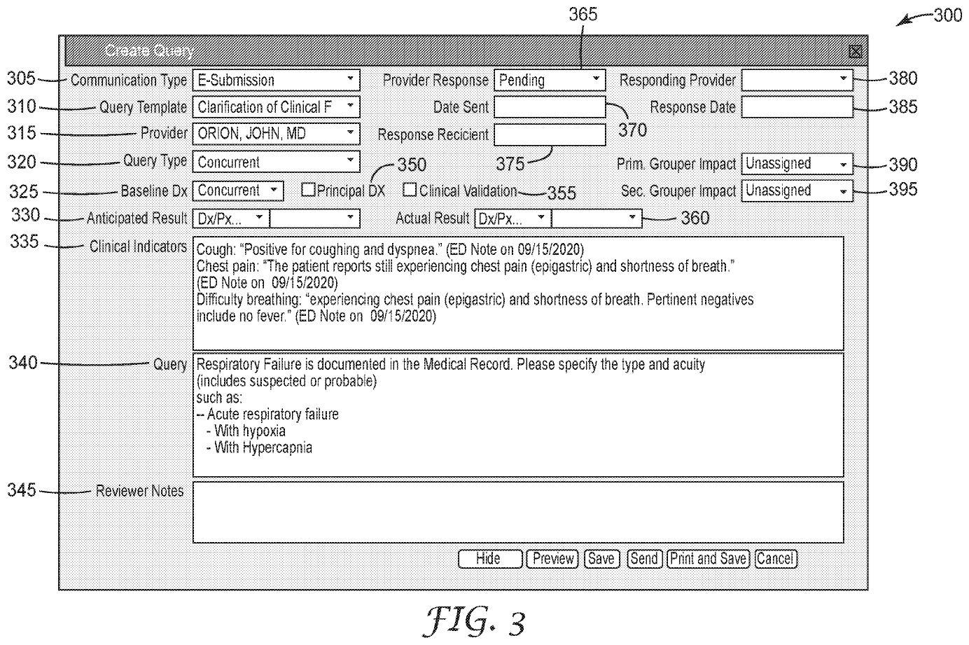

At operation 230, the baseline code is stored. This also may occur when the query is made available to the healthcare provider. In some implementations, a code as defined by the query is stored in datastore 115. For instance, the query can define a baseline Dx code. According to particular implementations, the baseline Dx code is the code for which the CDI specialist is querying the healthcare provider. For instance, the baseline Dx code can be selected from the existing codes associated with the patient encounter. If the CDI specialist is querying about a Dx code that is not present in the codeset, the CDI specialist can specify that the Dx code is “missing.” Although the baseline Dx is described in reference to a diagnosis, the baseline Dx may also include information pertaining to surgical procedures or other information related to the diagnosis. That is, a baseline Dx can be thought of as a baseline Dx/Px for the purposes described herein. Baseline Dx codes are described in more detail below in reference to FIG. 3.

At operation 235, one or more anticipated codes are stored. Like operations 225 and 230, this may also occur when the query is made available to the healthcare provider. The anticipated codes can also be stored in datastore 115. In some implementations, one or more anticipated result codes are the codes associated to one or more diagnoses the CDI specialist responsible for generating the query is expecting to result from the healthcare provider responding to the query. Anticipated result codes are described in more detail in reference to FIG. 3.

At operation 240, the system 100 determines whether a response has been received. Depending on the results of that determination, the system 100 may perform one or more incremental impact calculations. For instance, the system 100 can be configured to perform a realized incremental impact analysis, an incremental anticipated impact analysis, and incremental missed opportunity analysis. In general terms, an realized incremental impact analysis uses starting codesets, the actual resulting codes, and the baseline Dx value to retrospectively calculate the impact the query had on the provision of healthcare services at the time the query was generated, an incremental anticipated analysis determines the expected impact on the provision of healthcare services the query will have once the healthcare provider responds, and incremental missed opportunity impact analysis determines the potential negative impact to the provision of healthcare services for queries that are not answered. Each of the realized incremental impact analysis, incremental anticipated impact analysis, and incremental missed opportunity analysis are described in more detail below.

At operation 240, if the response has been received, the system 100 may perform a realized incremental impact analysis. For instance. a realized incremental impact analysis may begin when a first response providing further specificity with respect to a baseline diagnostic code is received at operation 245. In some implementations, an updated code specified by the CDI specialist in response to the first response is received at operation 245. The updated code may be entered into a user interface of the application 110 and stored at operation 245 in the data store 115. One example user interface is the user interface described in FIG. 3, below.

At operation 255, the application 110 executing on system 100 generates incremental impact summary data. The system 100 can generate the incremental summary data by executing an SQL stored procedure represented by this illustrative pseudo-code example:

If Actual Result = Not POA or Ruled-out
Then exit (don't generate an Incremental codeset)
Else continue
If Actual Result = POA
Then continue
If Actual Result = diagnosis code(s)
When multiple codes
Then insert as principal/secondary (positions #1 and #2)
When one code defined And Principal Dx is selected
Then insert into position #1
When one code defined And Principal Dx is not selected
Then insert into same position as baseline
If Baseline Dx = code
And code exists in linked codeset
Then remove the code
If Baseline Dx = missing
Then continue

As illustrated by the example pseudocode, the system 100 first checks to see if the “Actual Result” was not present on arrival at the time of the physician/patient encounter or was subsequently ruled out during the provision of medical care. If this condition is satisfied, then the system 100 does not generate an incremental codeset and the system 100 ceases the realized incremental analysis operation. If, however, the “Actual Result” was present on arrival the system 100 then compares the “Actual Result” to one or more diagnosis codes. If there is more than one diagnosis code, the system 100 determines which is the principal code and which is the secondary code. In some implementations, the system 100 receives user input that species a primary diagnosis and a secondary diagnosis. For instance, as will be described in more detail below, a user of the system can select a user interface component to indicate that a diagnosis is a principal diagnosis or a secondary diagnosis. According to some implementations, when the “Principal Dx” is selected, the system 100 defines the code as the principal diagnosis. In addition, when the “Principal Dx” is not selected, the system 200 defines the code as the baseline, or secondary diagnosis. The system 100 can then begin to modify the codeset. For instance, if the baseline diagnosis is the same as the code in the linked codeset, the system 100 can remove the code from the linked codeset. In this way, the system 100 can prepare a modified codeset for further analysis in the realized incremental impact analysis. Note that “Actual Result,” “Principal Dx,” and “Baseline Dx” can be human-readable information presented and/or provided in a user interface, such as the user interface in FIG. 3, described in more detail below. According to particular

According to particular implementations, after the above rules are executed and diagnosis codes in the codeset are updated (e.g., added or deleted), the SQL stored procedure returns the modified codeset in XML format. In some implementations, after the XML formatted codeset is generated, it is sent to a coding reimbursement system (CRS). The CRS responds with another XML document that includes grouper results. The system 100 may perform one or more additional steps, according to particular implementations including providing the grouper results to a web service. The web service may execute a series of SQL stored procedures to write the newly revised codeset to datastore 115. The new revision of the codeset is stored as an “incremental” data type. The system 100 may also update the query generated by the CDI specialist to include the new incremental codeset with a unique identifier generated by the web service. The resulting updated query is associated to the starting codeset and the incremental codeset. As a result, when an impact review screen is generated, the system calculates the differences between the starting codeset and the incremental codeset and displays the results. Examples of displayed results are described in more detail below.

Returning to operation 240, if the query has been saved but no response has been received, the system 100 may perform either anticipated incremental impact analysis or a missing incremental impact analysis. For instance, the system 100 can perform an anticipated incremental impact analysis or a missing incremental impact analysis by executing an SQL stored procedure represented by this illustrative pseudo-code example:

If Anticipated Result = Not POA or Ruled-out
Then exit (don't generate an Incremental codeset)
Else continue
If Anticipated Result = POA
Then continue
If Anticipated Result = diagnosis code(s)
When multiple codes
Then insert as principal/secondary (positions #1 and #2)
When one code defined And Principal Dx is selected
Then insert into position #1
When one code defined And Principal Dx is not selected
Then insert into same position as baseline
If Baseline Dx = code
And code exists in linked codeset
Then remove the code
If Baseline Dx = missing
Then continue

Again, “Anticipated Result,” “Principal Dx,” and “Baseline Dx” can be human-readable information presented in a user interface, such as the user interface in FIG. 3, described in more detail below. According to particular implementations, after the above rules are executed and diagnosis codes in the codeset are updated (e.g., added or deleted), the SQL stored procedure returns the modified codeset in XML format. In some implementations, after the XML formatted codeset is generated, it is sent to a CRS. The CRS responds with another XML document that includes grouper results. The system 100 may perform one or more additional steps, according to particular implementations including providing the grouper results to a web service. The web service may execute a series of SQL stored procedures to write the newly revised codeset to datastore 115. The new revision of the codeset is stored as an “anticipated” data type. The system 100 may also update the query generated by the CDI specialist to include the new incremental codeset with a unique identifier generated by the web service. The resulting updated query is associated to the starting codeset and the incremental codeset. As a result, when an impact review screen is generated, the system 100 calculates the differences between the starting codeset and the anticipated codeset and displays the results. Examples of displayed results are described in more detail below.

The primary difference between an anticipated incremental impact analysis and a missing incremental impact analysis is whether the query is identified as a “pending” query or assigned some other status that suggests no response to the query will be provided. This may occur when the query expires due to a healthcare provider not supplying an answering in the allotted time, or when the healthcare provider answer results an alternate diagnosis. For instance, at operation 270, when a completed query is saved, the system 100 can evaluate the response data to determine whether the response data is assigned a value consistent with “No Response,” “Unable to Determine/Unknown,” “No Codeable Response,” “Alternate Diagnosis/Response,” or other data values suggestive of a completed query containing no provider response. If the system 100 detects the presence of a completed query with no defined actual result in operation 270, the system 100 using application 110 may generate a missed opportunity impact summary at operation 275. At operation 275, the application 110 can send a data packet to an SQL stored procedure. In some implementations, the data packet includes the starting codeset that is linked to the completed query at operation 225. The system 100 can perform an anticipated impact analysis by executing an SQL store procedure represented by this illustrative pseudo-code example: represented by the previous illustrative pseudocode example.

FIG. 3 is a create query interface 300 generated by application 110 in response to selection of query 135 via interface 120. Options include a field for selecting a query template 310 that can be used to select from predefined templates according to a specific healthcare provider's workflow, and may be used to pre-populate other user interface components shown in FIG. 3. Additional options include a baseline diagnosis field 320 for selecting a baseline diagnosis, and a principal Dx check box 350 for indicating if the baseline diagnosis is a principal diagnosis. In addition, the query interface 300 also includes an anticipated result field 330 that allows the query to specify an anticipated code based on other information being provided to the query interface 300. In addition, the query interface 300 can include an actual result field 360 for indicating the resulting diagnosis or procedure code in response to the query based on a received response. It should be appreciated that the fields displayed in FIG. 3 can be used as input values for the decision making performed by the system 100, as described above. For instance, the “Actual Result” outlined in the pseudocode described above in relation to FIG. 2 can be defined by the value in the actual result field 360. Other values, such as “Principal Dx,” and “Baseline Dx” outlined above can be specified by the principal Dx field 350 and the baseline Dx field 325, respectively.

Query interface 300 also includes a clinical indicators field 335 for identifying symptoms, such as cough, chest pain, and difficulty breathing. The healthcare provider and date may be included with each symptom description. A query field 340 is provided for natural language query, or just a plain question from the user. As seen, options may make it easy for the healthcare provider to respond. A reviewer notes field 345 may also be provided if further information is desired to be communicated amongst other reviewers of the encounter, such as CDI managers, coders, concurrent coders.

Further fields for information housekeeping are provided and may be pre-filled by application 110 based on information stored in data store 115. Such fields may include a communication type 305, provider response status 365, query type 320, primary grouper query impact 390, secondary grouper query impact 395, responding provider 385, response recipient 375, date sent, 370, response date 385, and responding provider 380, which may be different than the response recipient 375. Many of these fields and other fields may be pre-filled by application 110 based on information in the data stored 115 and may also include drop down menus with options. Hide query, preview, save, send, print and save, and cancel buttons may also be provided.

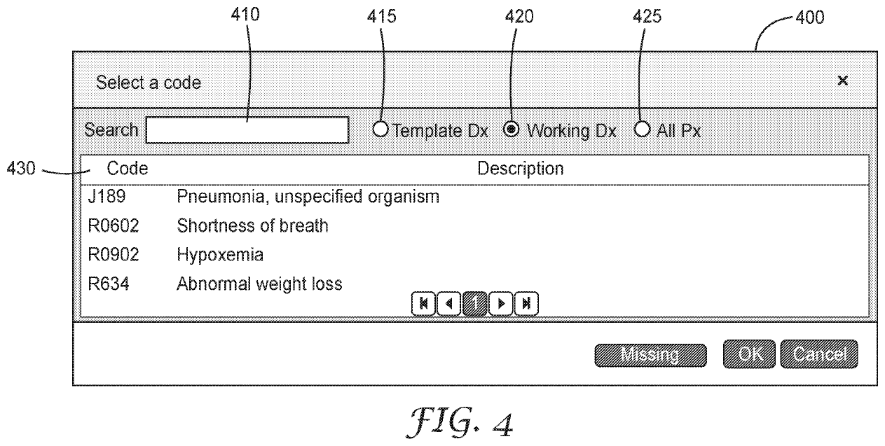

One example drop-down or pop-up is shown at 400 in FIG. 4 in response to selection of the Baseline Dx field 325. Pop-up 400 includes a search field 410 for use in searching, a template Dx button 415, a working Dx button 420, and an all Px button 425. Codes are also listed at 430 along with a description of each. The user may also set the code missing, if no code is currently associated. Baseline Dx and Actual Result are shown in addition to Primary and Secondary Grouper Impacts. If the Baseline Dx code selected is the Principle Diagnosis Code in the current codeset, then it will be checked. If the Baseline Dx code selected is not the Principle Diagnosis Code in the current codeset, then it is not checked. If Missing, then it is not checked. The user can override the checkbox. The selected code is then displayed in field 310.

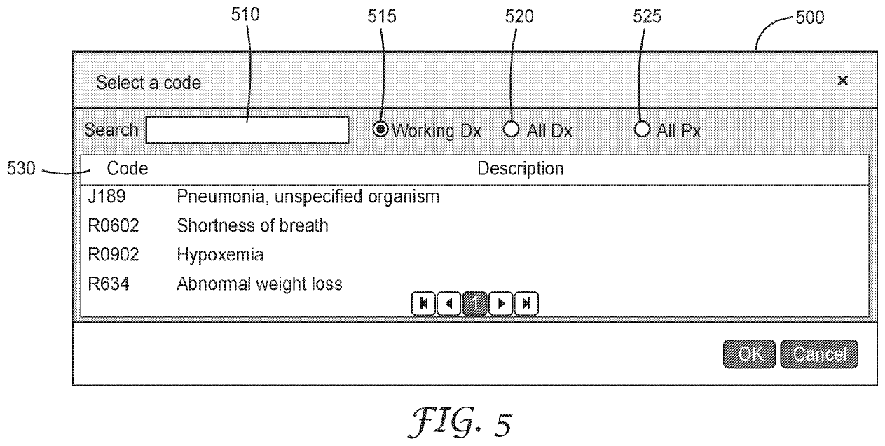

An example Actual Result pop-up is shown at 500 in FIG. 5. Pop-up 500 includes a search field 510 for us in searching, a working Dx button 515, an all Dx button 520, and an all Px button 525. Codes are also listed at 530 along with a description of each.

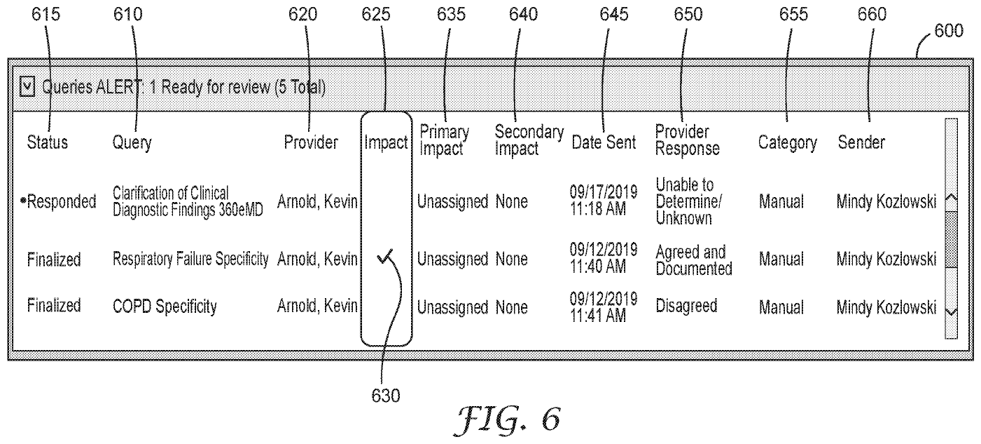

FIG. 6 is a query grid interface screen 600 that lists queries 610 and their status 615. Also listed for each query 610 are the corresponding provider 620, impact 625, checked impact 630, primary impact 635, secondary impact 640, date sent 645, provider response 650, category 655, and sender 660.

Checked impact 630 indicates that the query is linked to a final Dx/Px code.

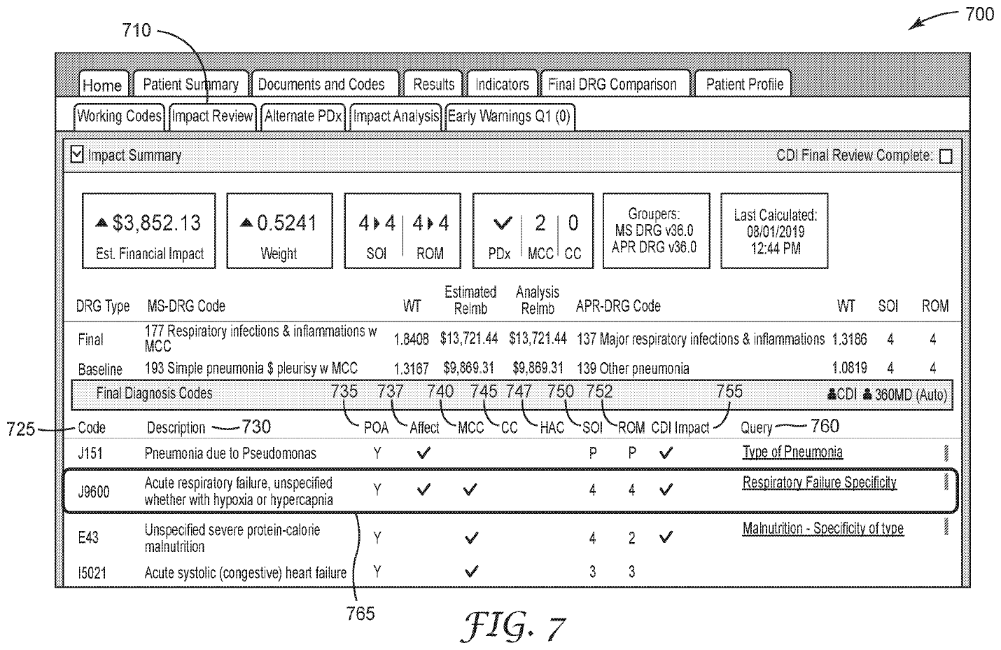

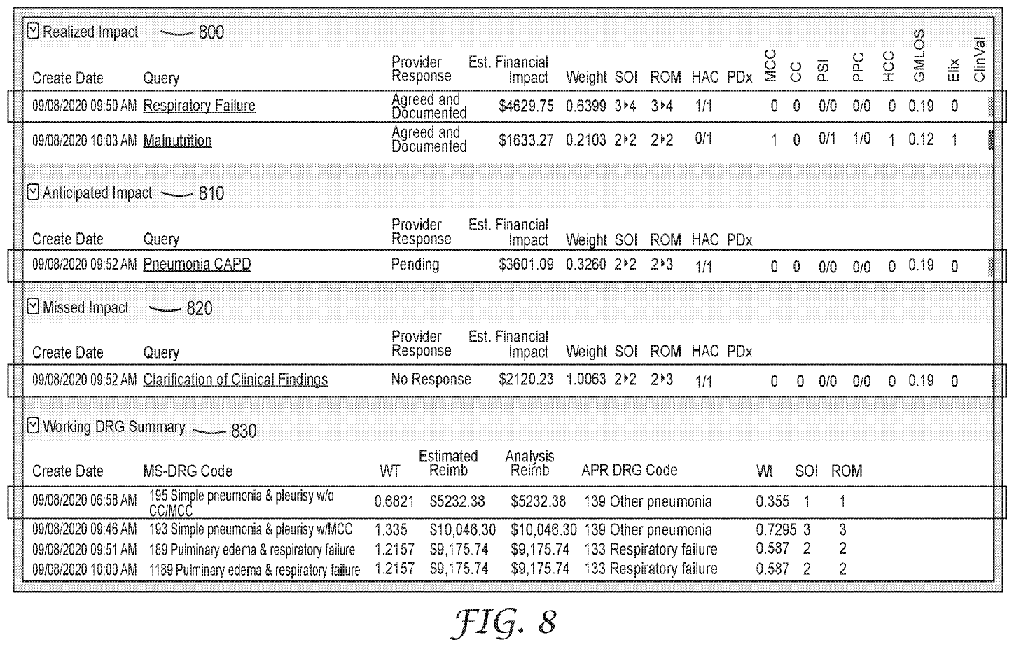

FIGS. 7 and 8 show respective interfaces show the effects of incremental queries during progression of a case at 700 and 800 involving a multi-day patient encounter. The updating of codes is illustrated corresponding to the following textual description of the progression of the case:

1. Pneumonia Automated CAPD generated

2. Working DRG1 MS-DRG 195 Pneumonia w/o MCC/CC (APR 139 SOI 2/ROM 1) created

3. Query for Acute Respiratory Failure (Associated with WDRG 1 and ‘Baseline Dx’ is ‘Missing’)

4. Query Malnutrition & Specificity (Associated with WDRG1 and ‘Baseline Dx is ‘Missing’)

5. Provider documented Respiratory Failure (‘Actual Result’ is J9600)

6. Working DRG 2 193 Pneumonia w/MCC (APR 139 SOI 3/ROM3) created

7. Provider documented Pneumonia due to Pseudomonas (‘Actual Result’ is J151)

8. Working DRG3 MS-DRG 177 Respiratory Infections & (APR 137 SOI 4/ROM 3) created

9. Provider documented Severe Malnutrition (‘Actual Result’ is E43)

10. Working DRG4 MS-DRG 177 Respiratory Infections & (APR 137 SOI 4/ROM 4) created

Interface 700 shows an impact review (tab 710) regarding a respiratory failure incremental query. A final MS-DRG code 715 and a baseline MS-DRG code 720 are illustrated, along with several other corresponding values, such as estimated financial impact, weight, SOI, ROM, PDx, MCC, CC, groupers, and last calculated time. Final diagnosis codes are also expanded as shown in a code column 725, descriptions 730, POA 735, Affect 737, MCC 740, CC 745, HAC 747, SOI 750, ROM 752, CDI Impact 755, and Query 760.

In interface 700, a selected code J9600 at 765 is shown in further detail in a working DRG summary 800 and incremental query impact summary 805 in FIG. 8. Working DRG summary 800 includes a selected DRG 1 at 810 for code 195— Pneumonia w/o MCC/CC with a weight of 0.6868, Reimb of $5,239.56, APR DRG Code of 139— Other Pneumonia, SOI of 2 and ROM of 1. A corresponding query summary is indicated at 815 for Respiratory Failure having an estimated financial impact of −4629.75, weight of −0.6299, SOI 2>3, ROM 1>3, MCC 1 and CC 0. These numbers show an overall positive impact of the incremental query.

Note that FIG. 7 shows other queries, including “Type of Pneumonia” and “Malnutrition-Specificity of Type.” These queries and corresponding codes are also listed in FIG. 8, and will be highlighted when selected in FIG. 7, just as the current selected code and corresponding information in FIGS. 7 and 8 are enclosed in a dark line. Highlighting or another attribute may be used to call attention to selected codes.

The Impact Review tab 710 automates much of the ROI and impact calculation of CDI contributions. Previously, determining the same impact took a few manual steps in different tabs, and you could not view the impact until a report was run.

FIG. 8 is an interface view showing a working summary of the effects of the various incremental impact summaries that the system 100 is configured to perform. The example interface presents information corresponding to realized incremental impact analysis pane 800, anticipated impact analysis pane 810, and incremental missed opportunity impact analysis pane 820. The interface may also present a comprehensive working DRG summary pane 830 to show an overview of the healthcare encounter.

For instance, in the working DRG summary pane 830, the first entry time stamped at 6:58 AM is a code for a patient presenting with pneumonia and/or pleurisy without comorbidities. The severity of illness (SOI) is rated at a 1, which is low, and the risk of mortality (ROM) is also rated at a 1, which is low. After a subsequent evaluation, time stamped 9:46 AM, the code was changed to simple pneumonia and pleurisy with comorbidities. This had an upward variance of the SOI and ROM from 1 to 3 due to a change in SOI in the new codeset. Soon thereafter, in the realized incremental impact analysis 800, a query was generated at 9:50 AM requesting that the physician evaluate the patient for respiratory failure. In response to the query, the medical provider indicated that the patient is suffering from respiratory failure which is shown by the “Agreed and Documented” label. As described above, because the medical provider provided a response, the system 100 is able to perform the realized incremental impact analysis as described elsewhere in this specification. For instance, the system can calculate a difference between the initial diagnosis of “simple pneumonia and pleurisy without commodities” and the actual diagnosis of “pulmonary edema and respiratory failure” to determine a realized incremental impact analysis. This difference may be a difference in weight, SOI, ROM, some combination of these, or other metrics.

FIG. 8 also shows the effects of other queries in the system as well. For instance, incremental anticipated impact analysis pane 810 illustrates results of an anticipated result that the patient has pneumonia and CAPD, pending a response from the physician. As shown in the example, the anticipated incremental impact shows no change in SOI and an increase in ROM from 2 to 3 based on an anticipated ROM of 3 in the anticipated codeset. In addition, the incremental missed opportunity impact analysis pane 820 illustrates an example result where a query concerning clarification of clinical findings went unanswered. This example incremental missed opportunity analysis may yield results indicating an increase ROM from 2 to 3 that reflects an increased ROM in a codeset that likely would have been provided had the query been responded to.

FIG. 8 also shows a number of other information which can be calculated as part of an incremental impact summary. For instance, “Est. Financial Impact” can represent a difference between the primary grouper (typically MS DRG) projected reimbursement amount from the linked codeset and the new incremental codeset. “Weight” can represent a difference between the primary DRG weight from the linked codeset and the new incremental codeset. “SOI” can represent a first number representing the SOI from the linked codeset and a second number representing the SOI from the incremental codeset. “ROM” can represent a first number representing the ROM from the linked codeset and a second number representing the ROM from the incremental codeset. “HAC,” or hospital acquired condition can represent information concerning whether a medical condition was acquired at the hospital. For example, using the query's Anticipated/Actual result diagnosis codes, the first number represents a count where the code resulted in a HAC avoidance. The second number represents the count of Anticipated/Actual Result diagnosis codes where the code confirmed a HAC.

“PDx,” or principal diagnosis is represented by a check or other visual indication when the Anticipated or Actual Result diagnosis code of an impactful query is in the incremental codeset in sequence position 1 (i.e., the primary diagnosis), and the linked codeset DRG does not match the incremental codeset DRG. “MCC,” or major complication or comorbidity, can represent information concerning the number of codes in the incremental codeset that are a major complication or comorbidity. For example, using the query's Anticipated/Actual result diagnosis codes, the system 100 can aggregate and present a count of those codes in the incremental codeset that are defined as an MCC. “CC,” or complication or Comorbidity, can represent information concerning the number of codes in the incremental codeset that are a complication or comorbidity. For instance, similar to the “MCC” field, using the query's Anticipated/Actual result diagnosis codes, the system 100 can aggregate and present a count of those codes in the incremental codeset that are defined as an CC.

“ClinVal,” or clinical validation is represented by a check or other visual indication when the intent of the query is to validate there is enough clinical evidence to support a code within the linked codeset. “PSI,” or patient safety indicator, can represent information concerning incidents of PSI avoidance and confirmed PSI. For example, using the query's Anticipated/Actual result diagnosis codes, the system can aggregate and present a first number representing a count where the code resulted in a PSI avoidance and a second number representing the count of Anticipated/Actual Result diagnosis codes where the code confirmed a PSI. “PPC,” or potentially preventable complication, can represent information concerning incidents of PPC avoidance and confirmed PPC. For example, using the query's Anticipated/Actual result diagnosis codes, the system can aggregate and present a first number represent a count where the code resulted in a PPC avoidance and a second number representing the count of Anticipated/Actual Result diagnosis codes where the code confirmed a PPC.

“HCC,” or hierarchical condition category, can represent information concerning an HCC. For example, using the query's Anticipated/Actual result diagnosis codes, the system 100 can aggregate and present a count of those codes in the incremental codeset that are defined as an HCC. “GMLOS,” or geometric length of stay, represents a difference between the linked codeset GMLOS and the incremental codeset GMLOS. According to particular implementations, an arrow points up for positive amounts and points down for negative amounts. “Elix,” or elixhauser comorbidity index, can represent inform concerning the elixhauser comorbidity index. For example, using the query's Anticipated/Actual result diagnosis codes, the system 100 can aggregate and present a count of those codes in the incremental codeset that are defined as an Elixhauser code.

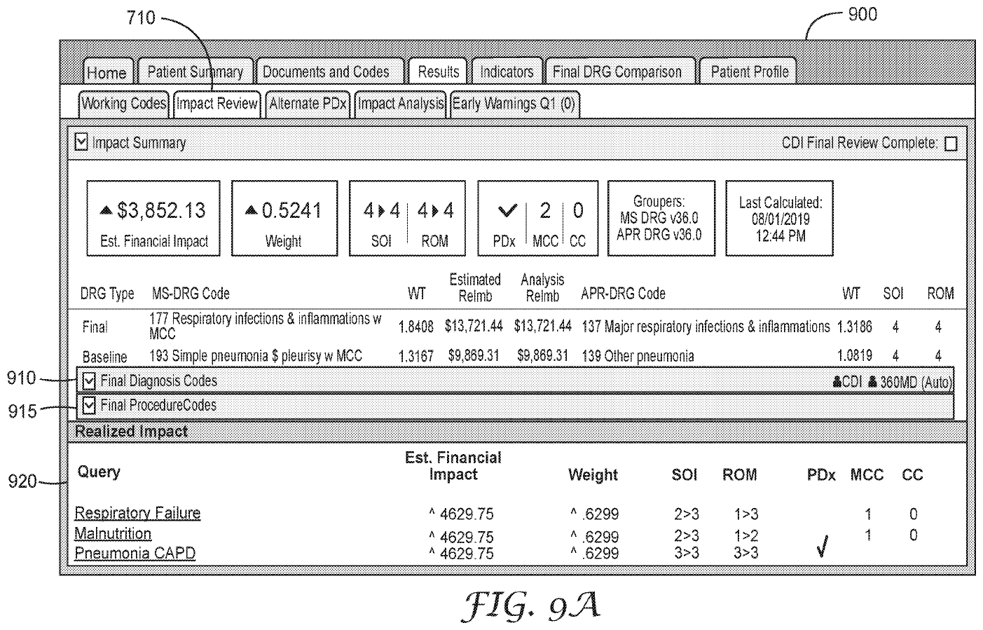

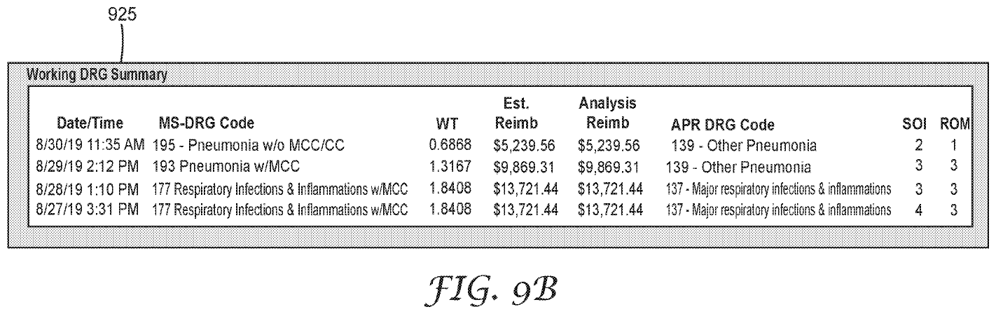

FIGS. 9A and 9B are a user interface view 900 corresponding to the impact review tab 710 with different portions of the view 900 expanded. The final diagnosis view 910 and final procedure codes view 915 are contracted in this view. An incremental query view 920 is expanded to show the three associated incremental queries and their impact on financial, weight, SOI, ROM, PDx, MCC and CC. A working DRG summary 925 is also provided in this view showing the dates of coding and corresponding information associated with the codes at the time of the coding

One big benefit of the Impact Review tab 710 is that it can track the CDI Impact when a visit changes from a Medical to a Surgical DRG. The previous method took manual “sandbox” analysis and then adjustments to show the impact reviewers had on the Final code set. The Impact Review tab tracks it automatically. In the example textual description of a patient encounter, a user can see the progression.

In the CDI Review workflow, the Impact Review subtab is under the Results tab. The Impact Review tab provides an accurate way of calculating the return on investment CDI actions had on the Final code set. Information appears on the Impact Review tab after Final coding is done and submitted.

The summary header shows the impact calculations. After the user makes changes, Save and Recalculate updates the information. Note that impacts go beyond financial and include quality impacts as well.

Changes in reimbursement (Est. Financial Impact) and Weight are shown. Arrows indicate if the values moved up or down.

SOI and ROM changes are noted. Arrows indicate the movement from the starting value on the left to the recalculated value on the right.

If a primary diagnosis code is linked to a query and affects the DRG, the PDx label has a check mark (the Affect column next to the diagnosis code in the section below has a check mark).

The MCC label displays a count of diagnosis codes with linked queries that have MCC checked in the section below.

The CC label displays a count of diagnosis codes with linked queries that have CC checked in the section below. The version of the groupers used appears as well as the date and time of the last calculation.

The DRG area shows the Baseline (when specified) and Final DRGs and related information. The Baseline row is calculated from any applicable codes in the Baseline Column.

In the Query column, a check mark appears if a query is linked to the code and the query template name appears as a link and can be selected to show the query details. When a visit is Final coded and submitted, the tab 710 shows the following: If the visit has only manual queries, the Final DRG and Final code set appear. If the visit has manual and automated specificity/acuity queries, the Final DRG, Final code set, and links to the Agreed and Documented automated queries appear. If the visit has no Final code set, the tab displays “No Records Found.” If the visit has only automated specificity/acuity queries, the DRGs section displays the specific changes that occurred between the calculated Baseline DRG, which is computed automatically using the unspecified code associated with the automated query, and the Final DRG with the specified code.

Calculating and displaying the incremental impact will allow accurate measurement of their CDI departments on the quality and completeness of their documentation.

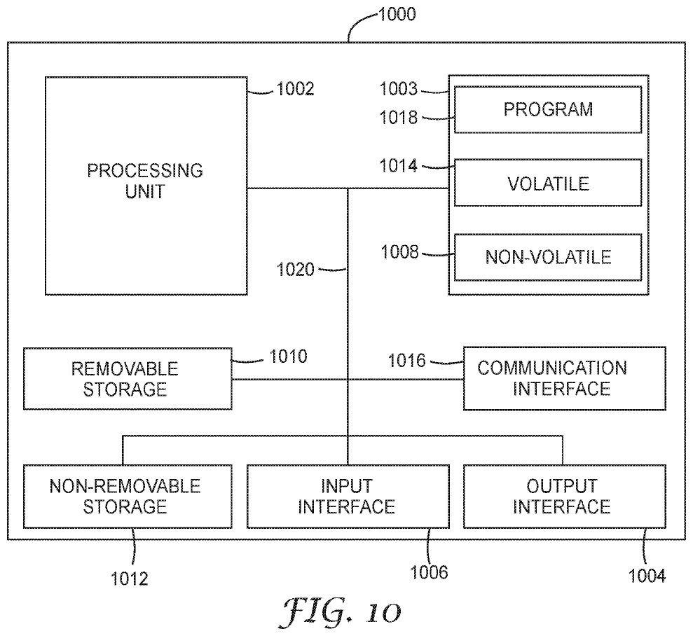

FIG. 10 is a block schematic diagram of a computer system 1000 to implement and manage the use of flexible workspaces and for performing methods and algorithms according to example embodiments. All components need not be used in various embodiments.

One example computing device in the form of a computer 1000 may include a processing unit 1002, memory 1003, removable storage 1010, and non-removable storage 1012. Although the example computing device is illustrated and described as computer 1000, the computing device may be in different forms in different embodiments. For example, the computing device may instead be a smartphone, a tablet, smartwatch, smart storage device (SSD), or other computing device including the same or similar elements as illustrated and described with regard to FIG. 10. Devices, such as smartphones, tablets, and smartwatches, are generally collectively referred to as mobile devices or user equipment.

Although the various data storage elements are illustrated as part of the computer 1000, the storage may also or alternatively include cloud-based storage accessible via a network, such as the Internet or server-based storage. Note also that an SSD may include a processor on which the parser may be run, allowing transfer of parsed, filtered data through I/O channels between the SSD and main memory.

Memory 1003 may include volatile memory 1014 and non-volatile memory 1008. Computer 1000 may include—or have access to a computing environment that includes—a variety of computer-readable media, such as volatile memory 1014 and non-volatile memory 1008, removable storage 1010 and non-removable storage 1012. Computer storage includes random access memory (RAM), read only memory (ROM), erasable programmable read-only memory (EPROM) or electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices, or any other medium capable of storing computer-readable instructions.

Computer 1000 may include or have access to a computing environment that includes input interface 1006, output interface 1004, and a communication interface 1016. Output interface 1004 may include a display device, such as a touchscreen, that also may serve as an input device. The input interface 1006 may include one or more of a touchscreen, touchpad, mouse, keyboard, camera, one or more device-specific buttons, one or more sensors integrated within or coupled via wired or wireless data connections to the computer 1000, and other input devices. The computer may operate in a networked environment using a communication connection to connect to one or more remote computers, such as database servers. The remote computer may include a personal computer (PC), server, router, network PC, a peer device or other common data flow network switch, or the like. The communication connection may include a Local Area Network (LAN), a Wide Area Network (WAN), cellular, Wi-Fi, Bluetooth, or other networks. According to one embodiment, the various components of computer 1000 are connected with a system bus 1020.

Computer-readable instructions stored on a computer-readable medium are executable by the processing unit 1002 of the computer 1000, such as a program 1018. The program 1018 in some embodiments comprises software to implement one or more methods of tracking incremental query effects and other method described herein. A hard drive, CD-ROM, and RAM are some examples of articles including a non-transitory computer-readable medium such as a storage device. The terms computer-readable medium and storage device do not include carrier waves to the extent carrier waves are deemed too transitory. Storage can also include networked storage, such as a storage area network (SAN). Computer program 1018 along with the workspace manager 1022 may be used to cause processing unit 1002 to perform one or more methods or algorithms described herein.

1. A computer implemented method comprising:
receiving, at one or more computer processors, a query soliciting further specificity with respect to a patient encounter with a healthcare provider;
generating, by the one or more computer processors, a unique identifier for an initial codeset specified by the query;
associating the unique identifier with the query;
storing, by the computer processors in a computer readable storage device, the initial codeset and the unique identifier with data describing the associated query;
receiving, by the one or more computer processors, a request for an impact summary, where the requested impact summary is one of a realized incremental impact summary, an anticipated incremental impact summary, and a missed opportunity incremental impact summary;
when the requested impact summary is a realized incremental impact summary, retrieving, from the computer readable storage device and by the one or more computer processors, the initial codeset associated with the query as specified by the unique identifier and evaluating, by the one or more computer processors, one or more updates to the initial codeset and a baseline diagnosis included in the query to determine an effect on the provision of medical care that the query had at the time the query was generated;
when the requested impact summary is an anticipated incremental impact summary, retrieving, from the computer readable storage device and by the one or more computer processors, the initial codeset associated with the query as specified by the unique identifier and evaluating, by the one or more computer processors, an effect on the provision of medical care that the query will have once the query is responded to;
when the requested impact summary is a missed opportunity incremental impact summary retrieving, from the computer readable storage device and by the one or more computer processors, the initial codeset associated with the query as specified by the unique identifier and evaluating, by the one or more computer processors, an effect on the provision of medical care had the query been responded to;
generating, by the one or more computer processors, the requested impact summary; and
presenting, on a display device communicatively coupled to the one or more computer processors, the generated impact summary.

2. The method of claim 1 wherein the effect of the requested impact summary comprises a change in severity of illness corresponding to each updated code.

3. The method of claim 1 wherein the effect of the requested impact summary comprises a change in risk of mortality corresponding to each updated code.

4. The method of claim 1 wherein the effect of the requested impact summary comprises a change in weight corresponding to each updated code.

5. The method of claim 1 wherein receiving the query comprises:
providing, on the display device communicatively coupled to the one or more computer processors, a query template for display, the query template including fields for a principal diagnosis, a response, and a natural language query; and
generating the query as a function of the natural language query and one of the first and further diagnoses from a first interface view that is generated by the one or more computer processors.

6. The method of claim 5 wherein the query template further comprises a clinical indicators field.

7. The method of claim 5 wherein the query template further comprises a field identifying a diagnosis that is the subject of the query, including the corresponding code, and a check box to indicate that the diagnosis is a principal diagnosis.

8. The method of claim 1 wherein the initial codeset and the unique identifier with data describing the associated query are stored in a relational database, and when a change to the codeset is detected, generating a new unique identifier and storing the changed codeset in the relational database with the new unique identifier.

9. The method of claim 1 wherein the realized incremental query impact summary comprises a list of queries for the patient encounter generated in response to a final code being identified, wherein the list of queries identifies a weight for each code, and a change of severity of illness and risk of mortality based on each query and the corresponding diagnosis, wherein the anticipated incremental query impact summary comprises one or more anticipated codes that would be generated in response to the query and a change of severity of illness and risk of mortality based on the query and the corresponding diagnosis when the query is responded to, and wherein the missed opportunity incremental impact summary comprises one or more anticipated codes that would be generated in response to the query and a change of severity of illness and risk of mortality based on the query and the corresponding diagnosis but were not generated because the query was unanswered.

10. A machine-readable storage device having instructions for execution by one or more computer processor of a machine to cause the one or more computer processors to perform operations to perform a method, the operations comprising:
receiving, by the one or more computer processors, a query soliciting further specificity with respect to a patient encounter with a healthcare provider;
generating, by the one or more computer processors, a unique identifier for an initial codeset specified by the query;
associating, by the one or more computer processors, the unique identifier with the query;
storing, by the computer processors in a computer readable storage device, the initial codeset and the unique identifier with data describing the associated query;
receiving, by the one or more computer processors, a request for an impact summary, where the requested impact summary is one of a realized incremental impact summary, an anticipated incremental impact summary, and a missed opportunity incremental impact summary;
when the requested impact summary is a realized incremental impact summary, retrieving, from the computer readable storage device and by the one or more computer processors, the initial codeset associated with the query as specified by the unique identifier and evaluating, by the one or more computer processors, one or more updates to the initial codeset and a baseline diagnosis included in the query to determine an effect on the provision of medical care that the query had at the time the query was generated;
when the requested impact summary is an anticipated incremental impact summary, retrieving, from the computer readable storage device and by the one or more computer processors, the initial codeset associated with the query as specified by the unique identifier and evaluating, by the one or more computer processors, an effect on the provision of medical care that the query will have once the query is responded to;
when the requested impact summary is a missed opportunity incremental impact summary retrieving, from the computer readable storage device and by the one or more computer processors, the initial codeset associated with the query as specified by the unique identifier and evaluating, by the one or more computer processors, an effect on the provision of medical care had the query been responded to;
generating, by the one or more computer processors, the requested impact summary; and
presenting, on a display device communicatively coupled to the one or more computer processors, the generated impact summary.

11. The device of claim 10 wherein the effect of the requested impact summary comprises one or more of a change in severity of illness, a change in risk of mortality, and a change in weight.

12. The device of claim 10 wherein operations for receiving the query from a comprises:
providing, on a display device communicatively coupled to the one or more computer processors, a query template for display, the query template including fields for a principal diagnosis, a response, and a natural language query; and
generating the query as a function of the natural language query and one of the first and further diagnoses from a first interface view that is generated by the one or more computer processors.

13. The device of claim 12 wherein the query template further comprises a clinical indicators field.

14. The device of claim 13 wherein the query template further comprises a field identifying a diagnosis that is the subject of the query, including the corresponding code, and a check box to indicate that the diagnosis is a principal diagnosis.

15. The device of claim 10 wherein the initial codeset and the unique identifier with data describing the associated query are stored in a relational database, and when a change to the codeset is detected, generating a new unique identifier and storing the changed codeset in the relational database with the new unique identifier.

16. The device of claim 10 wherein the realized incremental query impact summary comprises a list of queries for the patient encounter generated in response to a final code being identified, wherein the list of queries identifies a weight for each code, and a change of severity of illness and risk of mortality based on each query and the corresponding diagnosis, wherein the anticipated incremental query impact summary comprises one or more anticipated codes that would be generated in response to the query and a change of severity of illness and risk of mortality based on the query and the corresponding diagnosis when the query is responded to, and wherein the missed opportunity incremental impact summary comprises one or more anticipated codes that would be generated in response to the query and a change of severity of illness and risk of mortality based on the query and the corresponding diagnosis but were not generated because the query was unanswered.

17. A device comprising:
one or more computer processors;
a display device communicatively coupled to the one or more processors; and
a memory device communicatively coupled to the processor and having a program stored thereon for execution by the processor to perform operations comprising:
receiving, at the one or more computer processors, a query soliciting further specificity with respect to a patient encounter with a healthcare provider;
generating, by the one or more computer processors, a unique identifier for an initial codeset specified by the query;
associating, by the one or more computer processors, the unique identifier with the query;
storing, by the computer processors and on the memory device, the initial codeset and the unique identifier with data describing the associated query;
receiving, by the one or more computer processors, a request for an impact summary, where the requested impact summary is one of a realized incremental impact summary, an anticipated incremental impact summary, and a missed opportunity incremental impact summary;
when the requested impact summary is a realized incremental impact summary, retrieving, from the memory device and by the one or more computer processors, the initial codeset associated with the query as specified by the unique identifier and evaluating, by the one or more computer processors, one or more updates to the initial codeset and a baseline diagnosis included in the query to determine an effect on the provision of medical care that the query had at the time the query was generated;
when the requested impact summary is an anticipated incremental impact summary, retrieving, from the computer readable storage device and by the one or more computer processors, the initial codeset associated with the query as specified by the unique identifier and evaluating, by the one or more computer processors, an effect on the provision of medical care that the query will have once the query is responded to;
when the requested impact summary is a missed opportunity incremental impact summary retrieving, from the computer readable storage device and by the one or more computer processors, the initial codeset associated with the query as specified by the unique identifier and evaluating, by the one or more computer processors, an effect on the provision of medical care had the query been responded to;
generating, by the one or more computer processors, the requested impact summary; and
presenting, on the display device, the generated impact summary.

18. The device of claim 17 wherein the effect of the requested impact summary comprises one or more of a change in severity of illness, a change in risk of mortality, and a change in weight.

19. The device of claim 17 wherein operations for receiving the query from a comprises:
providing, the display device, a query template for display, the query template including fields for a principal diagnosis, a response, and a natural language query; and
generating the query as a function of the natural language query and one of the first and further diagnoses from a first interface view that is generated by the one or more computer processors.

20. The device of claim 19 wherein the query template further comprises a clinical indicators field, a field identifying a diagnosis that is the subject of the query, including the corresponding code, and a check box to indicate that the diagnosis is a principal diagnosis, and wherein the incremental query impact summary comprises a list of queries for the patient encounter generated in response to a final code being identified, wherein the list of queries identifies a weight for each code, and a change of severity of illness and risk of mortality based on each query and the corresponding diagnosis.
